(19) United States
(12) Patent Application Publication
(10) Pub. No.: US 2024/0094216 A1
(43) Pub. Date: Mar. 21, 2024

(54) ODD CHAIN FATTY ACID THERAPY FOR THE TREATMENT OF MITOCHONDRIAL DISEASES

(71) Applicant: UNIVERSITY OF SOUTH CAROLINA, COLUMBIA, SC (US)

(72) Inventor: NORMA FRIZZELL, COLUMBIA, SC (US)

(21) Appl. No.: 18/458,500

(22) Filed: Aug. 30, 2023

Related U.S. Application Data
(60) Provisional application No. 63/445,851, filed on Feb. 15, 2023.
(60) Provisional application No. 63/402,249, filed on Aug. 30, 2022.

(51) Int. Cl. G01N 33/68; A61K 31/25

(52) U.S. Cl. CPC G01N 33/6812; A61K 31/25; G01N 33/6848

(57) ABSTRACT

Methods disclosed herein are directed to treating a mitochondrial disorder in a subject. For instance, methods can be utilized to treat a mitochondrial disorder based on an amount of at least one metabolite measured in a biological sample of a subject.

### CROSS REFERENCE TO RELATED APPLICATIONS

This application claims filing benefit of U.S. Provisional Patent Application Ser. No. 63/402,249, filed on Aug. 30, 2022 and U.S. Provisional Patent Application Ser. No. 63/445,851, filed on Feb. 15, 2023, all of which are incorporated herein by reference.

### GOVERNMENT SUPPORT CLAUSE

This invention was made with government support under R56 NS116174-01 and R01 NS126851 awarded by National Institutes of Health (NIH). The government has certain rights in the invention.

### BACKGROUND

Mitochondrial diseases are a group of disorders or diseases that affect mitochondrial function. Mitochondrial disorders occur in about 1 in 5,000 people. Mitochondrial complex I deficiency is the most common cause of mitochondrial disease in children, accounting for approximately 30% of cases. The main function of mitochondria, which are in nearly every cell in an organism, is to produce energy to support organ function. When the mitochondrial function is disrupted, less energy is produced and cellular and organ dysfunction results.

Rare mitochondrial diseases occur as a result of a mutation in a gene encoding a component of the mitochondrial machinery. These poorly understood heterogeneous diseases lead to impaired mitochondrial function. Disrupted mitochondrial function limits adenosine triphosphate (ATP) production and predominantly affects energy demanding tissues, such as brain and muscle tissues. Leigh Syndrome is the most common of all mitochondrial diseases and is often caused by a mutation in a component of mitochondrial Complex I of the electron transport chain. This is the first entry point for electrons that are used to make ATP.

Leigh Syndrome is associated with a failure to thrive, muscle weakness, and brain lesions due to reduced mitochondrial energy production. Leigh Syndrome can be characterized by progressive neurodegeneration with bilateral necrotizing lesions of the brainstem and basal ganglia, resulting in lactic acidosis, ataxia, seizures, dystonia, and respiratory failure.

Mutations of Complex I subunits can lead to Leigh Syndrome. Complex I is a large (980 kDa) └-shaped protein assembly made of 45 peptides, with one flavin mononucleotide and eight iron-sulfur clusters. One of the first identified mutations of Complex I encoded was NDUFS4, a small (18 kDa) assembly protein. Ndufs4 mutations are associated with brainstem deterioration in humans. Further, similar clinical and neurological symptoms in humans with Leigh Syndrome have been observed in a NDUFS4 knockout (NDUFS4 KO) mouse. Interestingly, mutations in mitochondrial Complex I of the electron transport chain affect only certain tissues, such as neurological tissues.

Presently, there are no specific metabolite biomarkers for Leigh Syndrome and other mitochondrial disorders. Further, mitochondrial disease patients are currently prescribed regular vitamin and antioxidant supplements in most cases. Mutations in mitochondrial Complex I of the electron transport chain can also impact metabolites. Measuring the production of metabolites may serve as a biomarker to detect Complex I deficient mitochondrial disorders and provide an avenue for treatment.

What are needed in the art are independent measures that assess the amount of metabolite present in a subject with a Complex I mitochondrial disorder. For instance, a method to treat a mitochondrial disorder in a subject based on the amount of metabolite measured.

### SUMMARY

In general, the present disclosure is directed to methods of treating a mitochondrial disease in a subject. For instance, methods can be utilized to treat a mitochondrial disorder based on an amount of at least one metabolite measured in a biological sample of a subject.

Additionally, the present disclosure is directed to methods for determining an amount of a metabolite present in a subject. For instance, methods can include selecting a therapy based on an amount of a metabolite present in a biological sample; and administering the therapy to the subject if the amount of the metabolite present in the biological sample is less than a predetermined amount.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates a schematic of Tricarboxylic Acid Cycle Defects in the Ndufs4 Knockout model of Leigh Syndrome.

FIG. 2A illustrates the extracted ion chromatography for 2-Hydroxyglutarate enantiomers, D-2-HG and L-2-HG. The product ion masses measured for each analyte are indicated (m/z values).

FIG. 2B illustrates the extracted ion chromatography for ¹³C₅-D-2-HG and ¹³C₅-L-2-HG. The product ion masses measured for each analyte are indicated (m/z values).

FIG. 2C illustrates the extracted ion chromatography for lactate enantiomers, D-lactate and L-lactate. The product ion masses measured for each analyte are indicated (m/z values).

FIG. 2D illustrates the extracted ion chromatography for ¹³C₅-L-lactate. The product ion masses measured for each analyte are indicated (m/z values).

FIG. 2E illustrates the extracted ion chromatography for malate. The product ion masses measured for each analyte are indicated (m/z values).

FIG. 3A illustrates malate in NDUFS4 knockout mouse olfactory bulb. Data are mean±SE (*p<0.05; unpaired t-test with Welch's correction) with n=10/group.

FIG. 3B illustrates malate in NDUFS4 knockout mouse brainstem. Data are mean±SE (*p<0.05; unpaired t-test with Welch's correction) with n=10/group.

FIG. 4 illustrates L-lactate in NDUFS4 knockout mouse olfactory bulb. Knockout of NDUFS4 increased L-lactate 40% in OB (4.616 nmol/mg in KO versus 3.287 nmol/mg in WT).

FIG. 5 illustrates L-lactate in NDUFS4 knockout mouse brainstem. Knockout of NDUFS4 increased L-lactate 60% in BS (8.09 nmol/mg in KO versus 4.96 nmol/mg in WT). Data are mean±SE (*p<0.05; unpaired t-test with Welch's correction) with n=10/group.

FIG. 6 illustrates L-2-Hydroxyglutarate in NDUFS4 knockout mouse olfactory bulb. OB L-2-HG levels were unchanged with knockout of NDUFS4. Data are mean±SE (*p<0.05; unpaired t-test with Welch's correction) with n=10/group.

FIG. 7 illustrates L-2-Hydroxyglutarate in NDUFS4 knockout mouse brainstem. NDUFS4 KO BS strikingly showed a 26% decrease in L-2-HG (18.566 pmol/mg in KO versus 25.245 pmol/mg in WT). Data are mean±SE (*p<0.05; unpaired t-test with Welch's correction) with n=10/group.

FIG. 8 illustrates D-2-Hydroxyglutarate in NDUFS4 knockout mouse olfactory bulb. NDUFS4 KO OB showed a 57% decrease in D-2-HG (17.607 pmol/mg in KO versus 41.053 pmol/mg in WT). Data are mean±SE (*p<0.05; unpaired t-test with Welch's correction) with n=10/group.

FIG. 9 illustrates D-2-Hydroxyglutarate in NDUFS4 knockout mouse brainstem. Brainstem levels of D-2-HG were unchanged with knockout of NDUFS4. Data are mean±SE (*p<0.05; unpaired t-test with Welch's correction) with n=10/group.

FIG. 10 illustrates L-2-Hydroxyglutarate in NDUFS4 knockout mouse olfactory bulb.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, disclosed herein are methods of treating a mitochondrial disorder. More specifically, disclosed methods include analyzing a biological sample that includes metabolites of interest, so as to determine the presence or quantity of a specific metabolite of the subject. Metabolites, as referred herein, are small molecules, such as substrates for enzymes of metabolic pathways, intermediates of metabolic pathways, or products obtained by a metabolic pathway. The accumulation of certain metabolites contributes to detrimental metabolic reprogramming. In some embodiments, a metabolite has a molecular weight of from about 50 Da (Dalton) to about 200 Da, such as from about 75 Da to about 185 Da, such as from about 80 Da to about 160 Da, from about 90 Da to about 150 Da, from about 100 Da to about 145 Da, from about 110 Da to about 135 Da, or any range therebetween. The methods disclosed herein can be beneficially utilized in one embodiment for treating a mitochondrial disorder, which can be identified based on the amount of metabolite present in the biological sample.

The term “biological sample” as used herein refers to a sample obtained from a subject (e.g., a patient). For instance, a biological sample may include blood, serum, plasma, or tissue. In some embodiments, a metabolite can be measured in a biological sample, such as isolated lymphocytes, cerebrospinal fluid, brain tissue, urine, skin, biopsy material, tumor samples, or other tissue samples. It is understood that obtaining a biological sample from a subject refers to taking possession of said biological sample. For instance, the biological sample can be removed from the subject by a medical practitioner. In some embodiments, the biological sample can be analyzed by the medical practitioner or another person (e.g., a lab technician).

A “subject” as used herein refers to any mammal, such as a human or a non-human (e.g., mice). In some embodiments, the subject is a human and has been diagnosed or is suspected of having a mitochondrial disorder.

Disclosed methods can be beneficially utilized to determine the presence or quantity (e.g., amount) of metabolite in a biological sample and can be useful in determining potential genetic defects related to mitochondrial function. For instance, methods can be useful for determining a therapeutic treatment strategy for a subject based on the amount of a metabolite present in a cell or tissue sample.

The amount of a metabolite can be measured in a biological sample derived from a subject. For instance, brain tissue samples can be removed from a subject and prepared for analysis. In some embodiments, two or more biological samples can be obtained from a subject, and each sample can be separately analyzed (e.g., comparing the amount of metabolite present in each biological sample). The present disclosure has found this approach to be advantageous in differentiating normal tissue (e.g., standard levels of metabolite present) and abnormal tissue (e.g., reduced or increased levels of metabolite present). In some embodiments, the biological sample is subjected to a metabolite extraction.

In some embodiments, a total serum/plasma sample can be treated in order to precipitate the total metabolite, for example, using an agent that precipitates the metabolite. The precipitated agent can then be collected, e.g., by centrifugation, for further analysis of all collected metabolites of a sample, including L-2-hydroxyglutarate or D-2-hydroxyglutarate (Formula I). This approach may be beneficial as this can allow for a desired delay from the time of sample collection to metabolite analysis. For instance, serum or urine samples can be frozen with metabolite extraction and analysis carried out at a later time.

Metabolite extraction can be carried out in accordance with any suitable method known in the art. For instance, the method of extraction can be based on chemical and/or physical properties of the targeted metabolite. In some embodiments, the targeted metabolite to extract can be a polar metabolite. For instance, polar metabolites may include amino acids, polyamines, nucleic acids, sugars or small organic acids. In other embodiments, the targeted metabolite can be a non-polar metabolite. For instance, non-polar metabolites may include hydrophobic solvents, fatty acids, membrane lipids, polyketides, or phenolics.

Polar and non-polar metabolites may be extracted using an organic or inorganic solvent including dimethylsulfoxide (DMSO), alcohols, acetonitrile, dimethylformamide, tetrahydrofuran, or a combination thereof. In some embodiments, an ice-cold methanol solution is used to extract polar metabolites from the biological sample. In some embodiments, the methanol solution is from about 60% v/v methanol to about 95% v/v methanol, such as about 65% v/v methanol, such as about 70% v/v methanol, such as about 75% v/v, such as about 80% v/v methanol, such as about 85% v/v methanol, such as about 90% v/v methanol, or such as about 95% v/v methanol.

According to one embodiment, total metabolite quantification of a sample can be carried out via derivatization of the metabolite and determining the quantity of metabolite present in the sample. By way of example, whole metabolite sample (e.g., total metabolite separated from a serum sample) can be subjected to lysis. Following lysis, metabolite from the whole metabolite lysate can be used for analysis of L-2-hydroxyglutarate or D-2-hydroxyglutarate. For instance, the metabolite can be extracted to form a metabolite pellet. The dried pellet can then be derivatized, for instance, by chiral derivatization and dried (in vacuo). For instance, chiral derivatization agents may include diacetyl-1-tartaric anhydride (DATAN) or N-(o-toluenesulfonyl)-L-phenylalanyl chloride (TSPC). The extracted samples can be further separated to remove particulate matter and elute polar metabolites. Precursor ions can be prepared and analyzed using parallel reaction monitor (PRM) GC-MS/MS. The parent and daughter ion pairs monitored can include m/z 363→147 (D, L-2-HG), 368→152 (¹³C₅ D, L-2-HG), 305→89 (L-lactate), 308→92 (¹³C₃ L-lactate), 349→133 (L-malate), or any other specific ions produced by fragmentation of the above products.

The determination or quantification of metabolite present in a biological sample can be performed in one embodiment using isotope dilution mass spectrometry, e.g., using gas chromatography mass spectrometry (GC-MS) or liquid chromatography mass spectrometry (LC-MS). For instance, in one embodiment, the total quantification of L-2-hydroxyglutarate can be used to accurately measure the overall amount of L-2-hydroxyglutarate in brain tissue samples.

In one embodiment, the metabolite detected can be enantiomers of 2-hydroxyglutarate, such as L-2-hydroxyglutarate or D-2-hydroxyglurate. These metabolites are inhibitors of α-ketoglutarate-dependent dioxygenases and function by competing with demethylase for the α-ketoglutarate (αKG) binding site, such as ten-eleven translocation 2 (TET2) and Jumonji-C (JmjC) histone demethylases. Since 2-HG exists as both L and D enantiomers, in some embodiments, the derivatization of functional groups can be utilized to resolve the enantiomers by liquid chromatography-mass spectrometry (LC-MS). In some embodiments, chiral derivatization with DATAN is employed to discriminate between L-2-HG and D-2-HG. DATAN derivatization followed by LC-MS reduces the time, cost, and racemization of analytes compared to GC-MS methods. Lactate is often added to facilitate detection of low abundance metabolites in the preparations of standards for quantification. In some embodiments, lactate (Formula II) can be substituted with malate (Formula III) (whose hydroxyl group may also be derivatized by DATAN) to allow for the simultaneous detection of both 2-HG and lactate enantiomers. Surprisingly, methods in the present disclosure can be utilized to simultaneously detect 2-HG levels in parallel with lactate quantification, providing a reliable quantification of lactic acidosis in subjects, such as in brain tissue samples.

TABLE 1
Structure of Metabolites

| Metabolites | Structure: |
|---|---|
| 2-hydroxyglutarate (Formula I) | 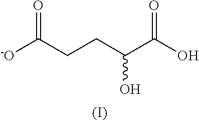 |
| Lactate (Formula II) | 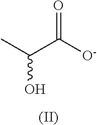 |
| Malate (Formula III) | 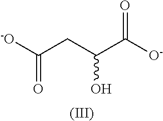 |

L-2-HG can be generated by malate dehydrogenase (MDH2) during metabolic acidosis, e.g. lactic acidosis, from αKG. The formation of L-2-HG by MDH2 under acidic conditions utilizes NADH rather than NAD⁺ which would be expected to be favored in the NDUFS4 KO model where the NADH/NAD⁺ ratio is elevated, and lactic acidosis is present. Accumulated L-2-HG can be removed via FAD⁺-dependent L-2-HG dehydrogenase (L2HGDH) converting L-2-HG back to αKG while generating FADH2.

In one embodiment, the metabolite can be D-2-HG. D-2-HG can be generated by the enzyme D-3-phosphoglycerate dehydrogenase (PHGDH). PHGDH is the first enzyme in the serine biosynthetic pathway but has a promiscuous activity to generate D-2-HG (and not L-2-HG) when αKG is abundant. This activity has been documented in select cancers, leading to the understanding of D-2-HG as an oncometabolite. Somatic mutations in isocitrate dehydrogenase (IDH) also result in specific D-2-HG accumulation, and these mutations occur in >80% of patients with diffuse WHO grade II-III gliomas. Specific D-2-HG metabolism has not been investigated in the NDUFS4 KO mouse.

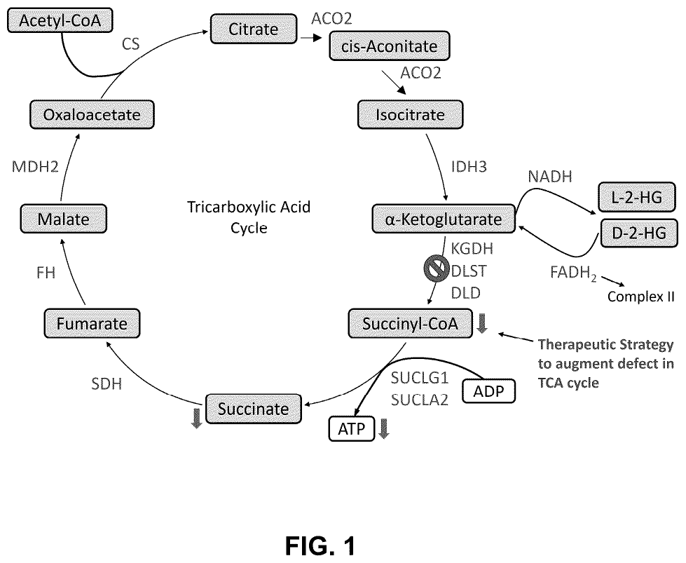

Turning to FIG. 1, dihydrolipoyllysine-residue succinyltransferase (DLST), a component of α-ketoglutarate dehydrogenase, activity is decreased in the Ndufs4 knockout mouse. Without wishing to be bound by theory, this results in decreased production of succinyl CoA and succinate, further exacerbating mitochondrial deficits. α-KG is shunted toward other metabolites including L and D 2-hydroxyglutarate enantiomers. Cycling between α-KG and 2-HG is beneficial in this disease model as it consumes NADH (which is present in excess in this model of Complex I deficiency) and generates FADH2 (which is useful to alternatively supply electrons to Complex II of the mitochondria). Overall, significantly lower levels of 2-HG enantiomers in this NDUFS4 KO mouse model has been detected compared to wild-type control mice. Given the defect at the α-KG axis, without wishing to be bound by theory, anaplerotic therapies may support the TCA cycle beyond this point, namely those entering as succinyl CoA, would benefit this mitochondrial Complex I deficits. Short odd chain fatty acids are metabolized to yield predominantly succinyl CoA.

In one embodiment, the metabolite detected can be a malate, such as L-malate. L-malate is a tricarboxylic acid (TCA) intermediate that is pivotal in transporting NADH from cytosol to mitochondria for energy production. In another embodiment, a metabolite detected can be a lactate, such as L-lactate or D-lactate. Lactate is an anion resulting from the dissociation of lactic acid and is an intracellular metabolite of glucose. Although the simultaneous measurement of these metabolites with 2-hydroxyglutarate enantiomers is not essential, it may be of benefit to track L-2-HG levels in parallel with metabolic acidosis, since lactate may facilitate its formation.

In some embodiments, methods provided herein involve measuring an amount of metabolite in a biological sample and comparing the metabolite level to an amount of a control sample so as to determine the amount of metabolite present in a subject. The absolute level of the metabolite can be quantified based on a standard curve derived from known amounts of a commercially available standard. The amount of control present can be an amount of the same metabolite in a control tissue or a control subject. For instance, the control amount of L-2-HG present can be derived from a normal subject. Herein, a normal subject refers to a control subject that is healthy and not diagnosed with a mitochondrial disorder. In another embodiment, the control amount can be derived from a subject with a mitochondrial disorder that presents with elevated or reduced metabolite levels. The control amount can be derived from a different tissue sample taken from the test subject. A finding that the metabolite level of a subject is decreased relative to a control amount by more than about 5% or some, such as more than about 10% or more, such as more than about 20% or more, such as more than about 30% or more, such as more than about 40% or more, or such as more than about 50% or more can determine a therapeutic strategy to treat a mitochondrial disorder in a subject.

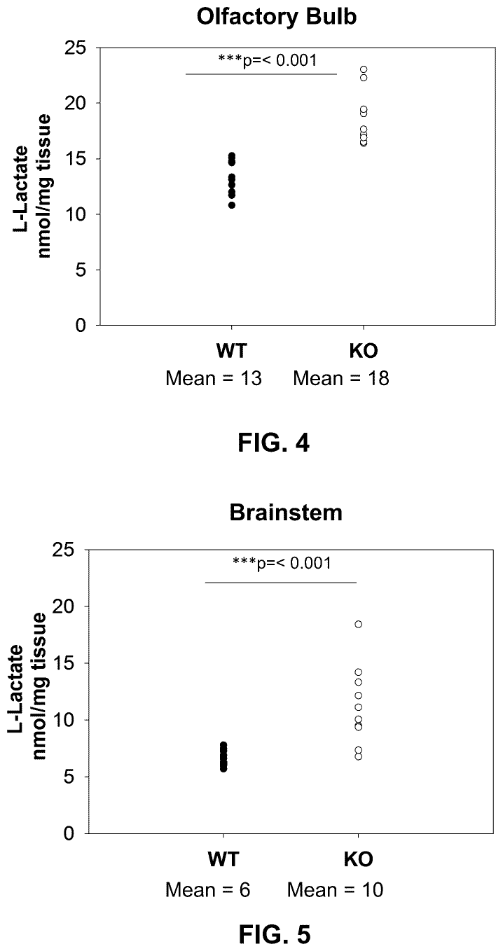

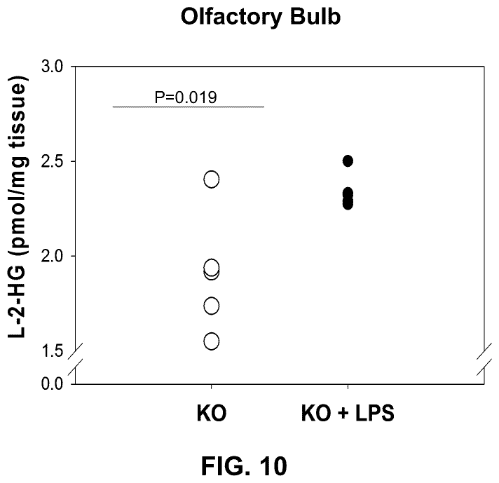

Surprisingly, it has been found that enantiomers, L-2-HG and D-2-HG, are significantly decreased in select brain tissue regions in a subject with Leigh Syndrome compared to a control subject. While it was expected that lactic acidosis might promote the formation of L-2-HG, and brain lactate is elevated (FIG. 4 and FIG. 5), L-2-HG and D-2-HG were either unchanged or decreased in the NDUFS4 KO disease state. In addition, both L-2-HG and D-2-HG were decreased in the serum of the NDUFS4 KO mouse (FIG. 10), despite increased lactate. Thus, 2-HG quantification can serve as a metabolic biomarker to detect Complex I deficient mitochondrial disorders, such as Leigh Syndrome. In some embodiments, a subject with Leigh Syndrome presenting with a decreased amount of metabolite compared to a control can be treated with one or more of (i) a triglyceride agent; and (ii) a vitamin. If desired, a subject can further be treated with an ester.

In some embodiments, the subject to be treated can have, is at risk for, or is suspected of having a mitochondrial disorder. The mitochondrial disorder may include Leigh Syndrome, Leber hereditary optic neuropathy, or other mitochondrial disorders. A subject having, at risk for, or suspected of having a mitochondrial disorder may experience a variety of symptoms such as fatigue, muscle weakness, metabolic stroke, brain lesions, seizures, cardiomyopathy, developmental or cognitive disabilities, diabetes, impairment of hearing, vision, growth, liver, gastrointestinal, or kidney function. The subject may experience said symptoms related to Leigh Syndrome at any age.

In some embodiments, a subject with reduced L-2-HG and/or D-2-HG levels may be treated by selecting an anaplerotic therapy. The anaplerotic therapy may be selected based on the level of metabolite present in the biological sample. For instance, the anaplerotic therapy may be a triglyceride, a short chain fatty acid, a short chain acylcarnitine, a ketone, a vitamin, or a combination thereof.

In one embodiment, the anaplerotic therapy may be a triglyceride. For instance, the triglyceride may be an odd-carbon chain fatty acid, e.g., isovaleric acid (5:0) heptanoic acid (7:0), nonanoic acid (9:0), pentadecanoic acid (15:0), or heptadecanoic acid (17:0). This may include the triglyceride form of these fatty acids, e.g. triheptanoin, the triglyceride of heptanoic acid (7:0).

In another embodiment, the anaplerotic therapy may be a short chain acylcarnitine. For instance, a short chain acylcarnitine may be selected from a group consisting of C3 (propionylcarnitine); C3-OH (hydroxypropionylcarnitine); C3:1 (propenoylcarnitine); C3-DC (C4-OH) (hydroxybutyrylcarnitine); C5 (valerylcarnitine); C5-M-DC (methylglutarylcarnitine); C5:1 (tiglylcarnitine); C5:1-DC (glutaconylcarnitine); or C5-OH (C3-DC-M) (hydroxyvalerylcarnitine or methylmalonylcarnitine). In one embodiment, the short chain acylcarnitine may be propionylcarnitine. In another embodiment, the short chain acylcarnitine may be butenylcarnitine.

In yet another embodiment, the anaplerotic therapy may be a short chain fatty acid. For instance, the short chain fatty acid may be propionic acid. In one embodiment, the short chain fatty acid may be valeric acid.

In another embodiment, the anaplerotic therapy may be a ketone. For instance, the ketone may be a C3 ketone. In one embodiment, the ketone is a C5 ketone.

In another embodiment, the anaplerotic therapy may be a vitamin. For instance, the vitamin may be selected from a group consisting of vitamin C, coenzyme Q10, B complex vitamins (e.g., thiamine B1 and riboflavin B2), α-lipoic acid, L-carnitine (e.g., Carnitor), creatine, and L-arginine.

If desired, an anaplerotic therapy may include an α-ketoglutarate ester selected from a group consisting of α-dimethyl ketoglutarate (DMKG), trifluoromethylbenzyl α-ketoglutarate (TFMKG), and octyl α-ketoglutarate (O-KG).

If desired, the anaplerotic therapy may include a combination of a triglyceride, a short chain fatty acid, a short chain acylcarnitine, a ketone, a vitamin, or an α-ketoglutarate ester. For instance, a subject may be treated with a short chain fatty acid in combination with a vitamin. In another embodiment, a subject may be treated with a short chain fatty acid in combination with a vitamin and an α-ketoglutarate ester.

In some embodiments, the anaplerotic therapy can be administered at a dose of at least about 0.05 g/kg body weight, 0.1 g/kg body weight, at least about 0.25 g/kg body weight, at least about 0.3 g/kg body weight, at least about 0.5 g/kg body weight, at least about 0.75 g/kg body weight, at least about 1.0 g/kg body weight, at least about 1.25 g/kg body weight, at least about 1.5 g/kg body weight, at least about 1.75 g/kg body weight, at least about 2.0 g/kg body weight, or at least about 3.0 g/kg body weight.

In some embodiments, the vitamin can be Vitamin B2 administered at a dose of 50-400 mg per os (po) daily. Other embodiments may include the above Vitamin B2 dose taken with coenzyme Q (as ubiquinol) at 2-600 mg po daily, or as ubiquinone at 10-2400 mg po twice-thrice daily. The dosing is dependent on whether the patient is pediatric or adult. In some embodiments, the vitamin, for instance, dose of at least about 0.1 mcg, at least about 0.25 mcg, at least about 0.5 mcg, at least about 0.75 mcg, at least about 1.0 mcg, at least about 1.25 mcg, at least about 1.5 mcg, at least about 1.75 mcg, at least about 2.0 mcg, at least about 2.5 mcg, or at least about 3.0 mcg.

In some embodiments, a vitamin supplement can be administered before, concurrent, or after administration of the short chain fatty acid. For example, the vitamin can be administered about 48 hours before, about 36 hours before, about 24 hours before, about 18 hours before, about 12 hours before, about 6 hours before, about 4 hours before, about 2 hours before administration of the short chain fatty acid. For instance, the vitamin can be administered about 48 hours after, about 36 hours after, about 24 hours after, about 18 hours after, about 12 hours after, about 6 hours after, about 4 hours after, about 2 hours after administration of the short chain fatty acid.

In some embodiments, an α-ketoglutarate ester supplement can be administered before, concurrent, or after administration of the short chain fatty acid and vitamin. For example, the α-ketoglutarate ester can be administered about 48 hours before, about 36 hours before, about 24 hours before, about 18 hours before, about 12 hours before, about 6 hours before, about 4 hours before, about 2 hours before administration of the short chain fatty acid and vitamin. For instance, the α-ketoglutarate ester can be administered about 48 hours after, about 36 hours after, about 24 hours after, about 18 hours after, about 12 hours after, about 6 hours after, about 4 hours after, about 2 hours after administration of the short chain fatty acid and vitamin.

In some embodiments, the α-ketoglutarate ester can be administered at a dose of at least about 100 mg, at least about 150 mg, at least about 200 mg, at least about 250 mg, at least about 3000 mg, at least about 500 mg, at least about 1000 mg, at least about 1500 mg, at least about 2000 mg, at least about 2500 mg, or at least about 3000 mg.

Regardless of whether the short chain fatty acid, vitamin, or α-ketoglutarate ester are administered before, concurrent, or after one another, the duration of therapy shall continue for as long as medically indicated or until a desired therapeutic effect is achieved. For instance, a subject can be treated as long as the mitochondrial disorder progression is delayed or inhibited based on the amount of metabolite present. In some embodiments, the short chain fatty acid and/or vitamin can be administered daily for a period of about 1 day, about 2 days, about 3 days, about 4 days, about 5 days, about 6 days, about 7 days, about 10 days, about 14 days, about 21 days, about 28 days, about 6 weeks, about 8 weeks, or longer than 8 weeks following first administration.

The present disclosure may be better understood with reference to the following examples.

### EXAMPLES

### Materials and Methods

Unless otherwise noted, all chemicals and analytical grade solvents were obtained from Millipore Sigma (Burlington, MA), ThermoFisher Scientific (Waltham, MA) or Cambridge Isotope Laboratories (Andover, MA) unless stated otherwise.

Animal care and use procedures were conducted in accordance with the Guide for the Care and Use of Laboratory Animals and approved by the University of South Carolina Animal Care and Use Committee. Mice on a mixed 129/Sv:C57BL/6 genetic background were maintained for breeding at the University of South Carolina School of Medicine Department of Laboratory Animal Resources. Male and female mice were sacrificed following isoflurane anesthesia and surgical decapitation at 7 weeks. The brain was removed and the tissue dissected. Blood was immediately collected and allowed to clot at room temperature (RT) followed by centrifugation at 400 g for 10 min at RT. The serum supernatant was collected and flash-frozen in liquid nitrogen.

Quantification of 2-hydroxyglutarate enantiomers and L-lactate was performed following adaptation of standard methods known in the art. All standards and samples contained the same final concentration of the internal standards (IS). L-lactate ¹³C₃ (50 μM, CLM-1579, Cambridge Isotope Laboratories, Andover, MA) and ¹³C₅ D,L-2-hydroxyglutarate (5 μM, CLM-10351, Cambridge Isotope laboratories) were prepared in 80% (v/v) methanol to a final volume of 200 μL. Standard curves containing D-2-HG and L-2-HG enantiomers in the micromolar range (MilliporeSigma; 61382 (D-2-HG), 61313 (L-2-HG)), and L-Lactate (Acros Organics, Carslbad, CA) in the millimolar range, were constructed based on values derived from pilot studies in serum. L-malate (100 nM) was added to all standards as a constant to aid derivatization of low 2-HG concentrations. All standards were dried by centrifugal evaporation prior to derivatization.

Metabolites were extracted from 50 μl serum containing the spiked internal standards following addition of ice-cold methanol 80% (v/v) and centrifugation at 3220 g for 20 min at 4° C. The supernatants were transferred to new 1.5 mL tubes and dried by centrifugal evaporation. The dried metabolites and standards were derivatized upon addition of 50 μL diacetyl-1-tartaric anhydride (DATAN), prepared fresh as a 50 mg/mL solution in acetonitrile:acetic acid (4:1, v/v). The derivatization reaction was allowed to proceed at 70° C. for 2 hr. Tubes were cooled to room temperature and centrifuged briefly before final dilution with 50 μL acetonitrile:acetic acid (4:1, v/v). After vortexing, the samples were briefly centrifuged, and the supernatant transferred to a microcentrifuge tube prior to LC-MS analysis.

LC separation was performed on a Thermo Vanquish Flex liquid chromatography system (ThermoFisher Scientific) using a PEEK coated SeQuant ZIC HILIC column (MilliporeSigma). The column was 2.1 mm by 150 mm with 3.5 μm particles and was preceded by an equivalent 20 mm long guard column. The column was maintained at 25° C. Following a 1 μl sample injection, isocratic elution was performed with 15% mobile phase A (10% 200 mM formic acid:90% LC-MS grade water) and 85% mobile phase B (10% 200 mM formic acid:90% acetonitrile), at a total flow rate of 200 μl/min. The 200 mM formic acid was titrated to pH 3.25 with ammonium hydroxide prior to inclusion in the mobile phase. Single injections of enantiomeric pure standards for either D-2-HG or L-2-HG confirmed their separation and retention times by LC analysis.

Negative ion electrospray mass spectra were acquired on a Thermo Q-Exactive™ HF-X Quadrupole-Orbitrap™ Mass Spectrometer performing parallel reaction monitoring (PRM). Precursor ions (363, 368, 305, 308 or 349 Da) were isolated by the quadrupoles and fragmented in the HCD cell at 10 eV. The Orbitrap resolution used for PRM was 30,000. The source capillary temperature was 300° C., other MS source settings were sheath gas flow: 45, auxiliary gas flow: 10, sweep gas flow: 2, spray voltage: 2.5 kV, funnel RF level: 20, auxiliary gas temperature: 400° C. XCalibur™ 4.2 software (ThermoFisher Scientific) was used to construct extracted ion chromatograms of the transitions 363→147 (D, L-2-HG), 368→152 (¹³C₅ D, L-2-HG), 30→589 (L-lactate), 308→92 (¹³C₃ L-lactate) and 349→133 (L-malate). The area of the analyte peaks were normalized to the area of the ¹³C internal standard for that specific analyte to obtain peak area ratios. These ratios accounted for differences in the responses of the 2-HG enantiomers. Duplicate sample injections were used to confirm peak area ratios and the mean peak area ratio from these was used to determine sample concentration. The concentration was determined from the standard curves and the mass of each analyte was calculated based on the 100 μl solution volume. The mass of each analyte in 50 μl serum was extrapolated to calculate the serum concentration of each analyte in micromolar (2-HG) or millimolar (L-lactate) levels.

### Results

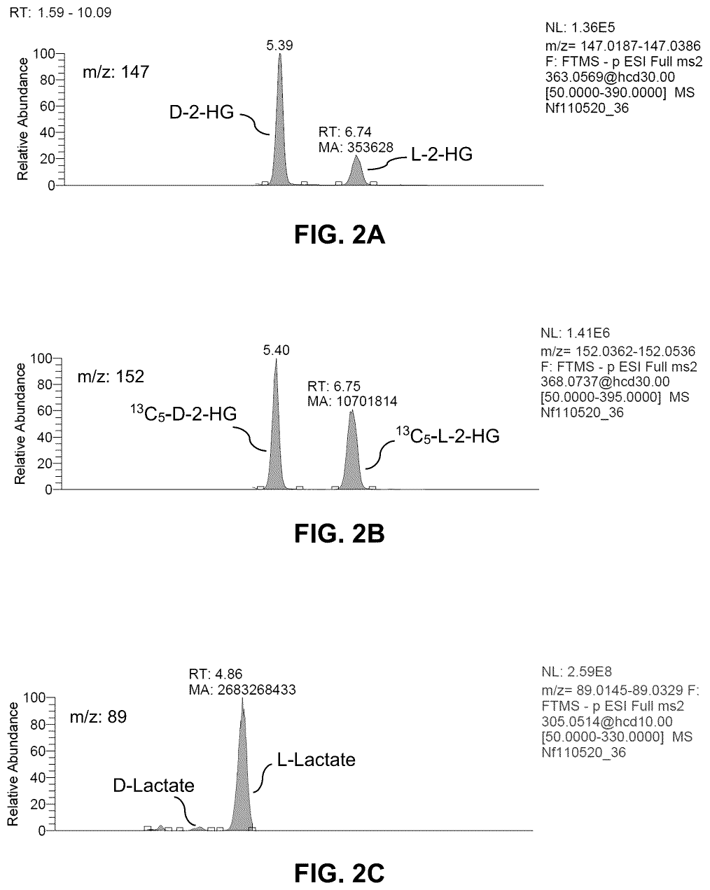

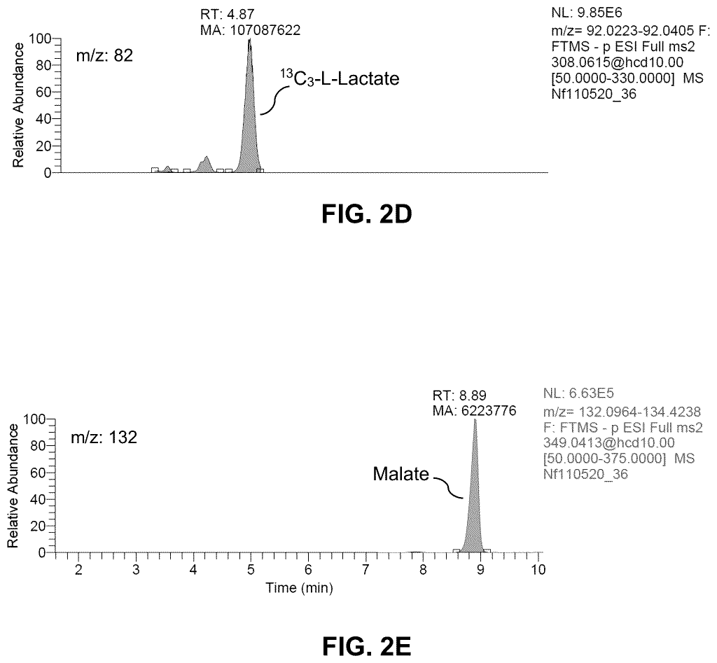

To determine if L-malate could be used to replace lactate as a carrier for the 2-HG standard derivatization, 2-HG and lactate standards were derivatized and their resolution was confirmed by LC-MS. Further, the added malate was also detected based on its unique fragmentation pattern. Malate was only added to the standards to improve derivatization as the samples contained endogenous malate. Both enantiomers of 2-HG, as well as L-lactate and L-malate, were monitored for all subsequent analyses with tissue samples. Representative extracted ion chromatograms from the derivatization of WT mouse serum shows all enantiomers were detected with separate retention times by LC (FIG. 2A-2E). The unique masses of product ions derived from the fragmentation of these metabolites by mass spectrometry were used to confirm their identity by LC-MS. The derivatized L-malate was also detected (FIG. 2E). D-lactate was not detected as serum D-lactate does not accumulate to appreciable amounts in vivo. Isotope dilution mass spectrometry was performed to determine the concentration of the metabolites of interest. All samples and standards were spiked with a fixed amount of isotopic D,L-2-HG and L-Lactate. Peak area ratios were calculated for the known standards and used to calculate the absolute metabolite amounts.

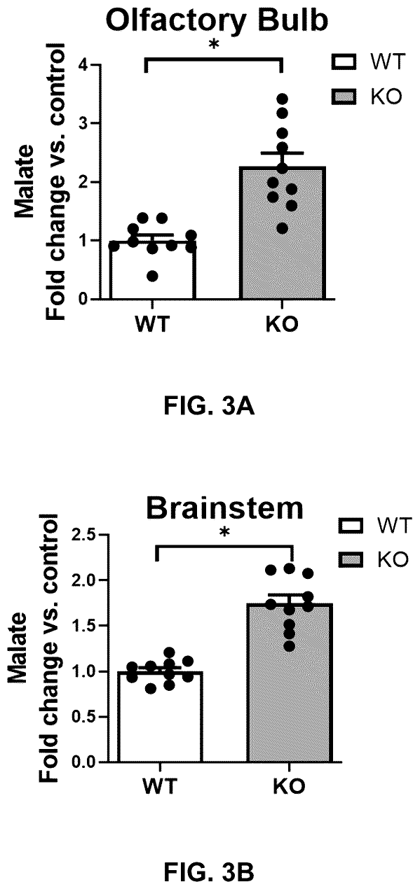

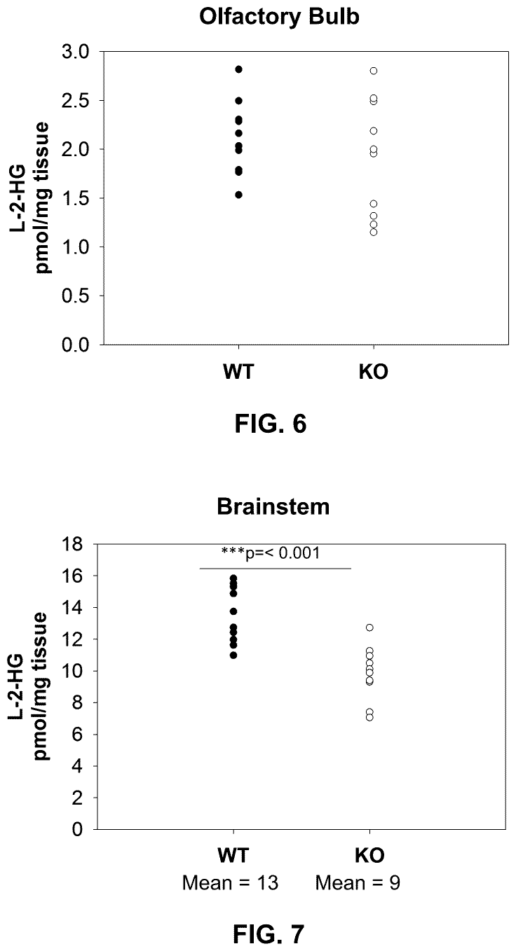

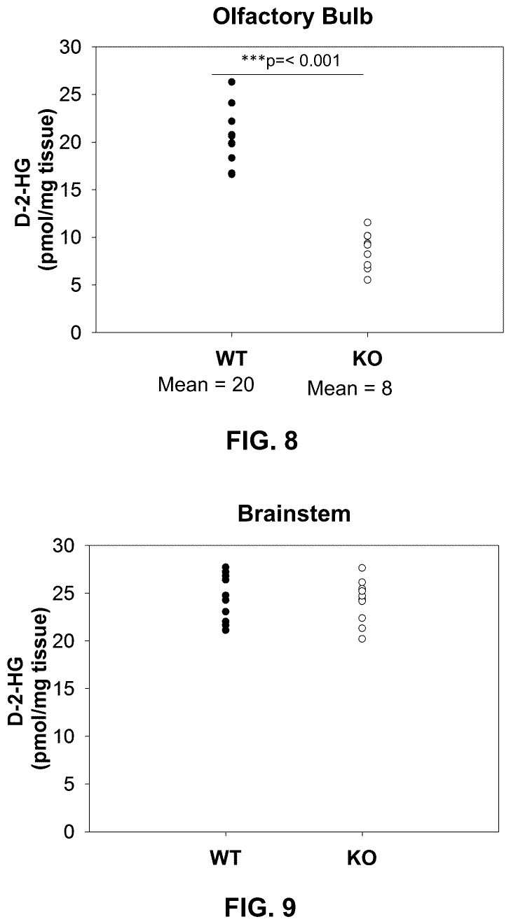

P42 WT and NDUFS4 KO mouse olfactory bulb (OB) and brainstem (BS) were analyzed following DATAN derivatization by LC-MS to determine 2-HG enantiomer and L-lactate levels. NDUFS4 KO mice had increased malate in the OB and BS compared to WT controls (FIG. 3A; FIG. 3B). This result supports previous analyses of malate levels by GC-MS. In addition, L-lactate increased by 60% in the BS (8.09 nmol/mg in KO versus 4.96 nmol/mg in WT) and 40% in the OB (4.616 nmol/mg in KO versus 3.287 nmol/mg in WT) with knockout of NDUFS4 (FIG. 4; FIG. 5). Quantification of L-2-hydroxyglutarate in the olfactory bulb demonstrated no change, but a striking 26% decrease in brainstem L-2-HG was detected in NDUFS4 KO versus WT (FIG. 6; FIG. 7). In contrast, a 57% decrease was measured in olfactory bulb D-2-HG and no change in brainstem D-2-HG with knockout of NDUFS4, suggesting region-specific differences in metabolite dynamics (FIG. 8; FIG. 9).

Next, the levels of lactate and 2-hydroxyglutarate in P49 WT and NDUFS4 KO mouse serum were analyzed to quantify systemic metabolite levels. While the level of L-lactate increased 40% as expected, the levels of L-2-HG were decreased 28% in NDUFS4 KO serum versus WT. In addition, a remarkable 61% decrease was quantified in D-2-HG in NDUFS4 KO serum versus WT. While the decrease in L-2-HG or D-2-HG was surprising in select brain regions, the serum measurements validate these surprising findings and suggest that both enantiomers are selectively reduced in tissues of the NDUFS4 KO versus WT.

### Discussion

Methods disclosed herein permits the simultaneous detection of lactate and 2-hydroxyglutarate enantiomers. The present disclosure measured increased malate and an ˜1.5-fold increase in lactate with knockout of NDUFS4, which confirmed the expected lactic acidosis throughout the body. The confirmation of increased lactic acidosis in these regions affected by the pathology underscored the premise for the proposed L-2-HG measurement, as it was also expected to increase. Surprisingly, knockout of NDUFS4 did not alter basal levels of L-2-HG in the OB, where previously reduced activity of the α-ketoglutarate dehydrogenase complex had been detected. Unexpectedly, L-2-hydroxyglutarate decreased 26% in the BS. These data, while striking, also confirmed that L-2-HG does not appear to have a significant role in regulating histone demethylases. Strikingly, D-2-HG levels were quantified in parallel with L-2-HG, and noticed levels of D-2-HG were markedly reduced in the OB, with a 57% decrease with knockout of NDUFS4. In contrast to L-2-HG, D-2-HG levels were unchanged in the brainstem.

WT and NDUFS4 KO serum was used to quantify systemic metabolite levels. While a 40% increase in L-lactate was measured, the levels of L-2-HG were decreased 28%, and the levels of D-2-HG were decreased 61% in NDUFS4 KO serum versus. Since serum findings were consistent for both enantiomers and were significantly reduced in the knockout versus wild-type, this suggests that this reduction may be a specific biomarker for this Complex I deficit.

As the metabolites were reduced rather than increased, this suggests that basal levels of these metabolites may be utilized. The fate of αKG was not detected in methods disclosed herein. It can be hypothesized that acidosis favors L-2-HG generation, and this may occur, but the subsequent L2HGDH mediated reduction to αKG may be favored to generate precious FADH2, which can supply electrons to Complex II. FADH2 offers the benefit of bypassing the NDUFS4 KO-induced Complex I bioenergetic deficit by funneling electrons to a functional complex that delivers them to ubiquinone for subsequent transit to Complex III. 2-hydroxyglutarate, as a source of FADH2 for Complex II, may in part explain why oxidative phosphorylation continues to be supported in NDUFS4 KO mice with the loss of Complex I.

It remains unclear why region-specific differences were observed in the brain region-specific reductions in these metabolites. Without being bound by theory, it is interesting to speculate that variable expression of the associated dehydrogenases, L2HGDH and D2HGDH, is associated with the varied expression of 2-HG enantiomers. Alternatively, the differences in 2-HG levels may reflect region-specific metabolic dynamics with the 1.5-fold increase in L-lactate and >5-fold increase in L-2-HG in the WT and NDUFS4 KO brainstem compared to the olfactory bulb. The decrease in serum 2-HG levels with increased L-lactate suggests that 2-HG may be used systemically to maintain limited mitochondrial oxidative phosphorylation. Therefore, supplementation with the α-ketoglutarate ester, DMKG, may provide systemic benefits to NDUFS4 KO mice.

In summary, the robust reduction in 2-HG enantiomers in brain regions and serum of the NDUFS4 KO mouse suggest that these metabolites may have as yet unappreciated roles in supporting metabolism through alternative delivery of electrons derived from αKG oxidation. Further analyses of L2HGDH and D2HGDH enzyme activity coupled with the assessment of rates of production will be necessary to determine the synthesis and fate of these metabolites.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method of treating a mitochondrial disorder, comprising:
i. determining an amount of a metabolite present in a biological sample of a subject;
ii. selecting an anaplerotic therapy based on the amount of the metabolite present in the biological sample; and
iii. administering the anaplerotic therapy to the subject if the amount of the metabolite present in the biological sample is less than a predetermined amount.

2. The method of claim 1, the biological sample comprising blood, serum, plasma, or tissue.

3. The method of claim 1, wherein the metabolite comprises L-2-hydroxyglutarate, D-2-hydroxyglutarate, D-lactate, or L-lactate.

4. The method of claim 1, wherein the mitochondrial disorder comprises Leigh Syndrome, Leigh Syndrome with Complex I mutations, or Lebers Hereditary neuropathy.

5. The method of claim 1, wherein the anaplerotic therapy comprises a triglyceride, a short chain fatty acid, a short chain acylcarnitine, a ketone, a vitamin, or a combination thereof.

6. The method of claim 5, wherein the short chain fatty acid comprises propionic acid or valeric acid.

7. The method of claim 5, wherein the short chain acylcarnitine comprises propionylcarnitine or butenylcarnitine.

8. The method of claim 5, wherein the short chain acylcarnitine comprises hydroxypropionylcarnitine, propenoylcarnitine, hydroxybutyrylcarnitine, valerylcarnitine, methylglutarylcarnitine, tiglylcarnitine, glutaconylcarnitine, hydroxyvalerylcarnitine, or methylmalonylcarnitine.

9. The method of claim 5, wherein the triglyceride comprises triheptanoin.

10. The method of claim 5, wherein the ketone comprises a C3 ketone or a C5 ketone.

11. The method of claim 5, wherein the vitamin is selected from vitamin C, coenzyme Q10, B complex vitamins, α-lipoic acid, L-carnitine, creatine, and L-arginine.

12. The method of claim 1, wherein the anaplerotic therapy further comprises at least one α-ketoglutarate ester.

13. The method of claim 12, wherein the α-ketoglutarate ester is selected from α-dimethyl ketoglutarate, trifluoromethylbenzyl α-ketoglutarate, and octyl α-ketoglutarate.

14. The method of claim 1, wherein the biological sample is analyzed using liquid chromatography mass spectrometry.

15. The method of claim 1, further comprising contacting a derivatization standard to the biological sample.

16. The method of claim 14, wherein the derivatization standard is diacetyl-1-tartaric anhydride (DATAN) or malate.

17. The method of claim 1, wherein the predetermined amount is the amount of metabolite present in the same biological sample from a non-mitochondrial disorder subject.

18. The method of claim 1, wherein the amount of the metabolite present is less than about 20% of the predetermined amount.

19. The method of claim 5, wherein the short chain fatty acid and the vitamin are administered concurrently or sequentially.

20. The method of claim 1, wherein the anaplerotic therapy is administered at a dose of about 0.1 g/kg to about 2.0 g/kg.

21. The method of claim 5, wherein the short chain fatty acid is administered at a dose of about 0.1 g/kg to about 2.0 g/kg.

22. The method of claim 5, wherein the vitamin is administered at a dose of about 0.1 mcg to about 1.5 mcg.

23. The method of claim of claim 5, wherein the α-ketoglutarate ester is administered at a dose of about 100 mg to about 3000 mg.

24. A method for determining an amount of a metabolite present in a subject, comprising:
i. selecting a therapy based on an amount of a metabolite present in a biological sample; and
ii. administering the therapy to the subject if the amount of the metabolite present in the biological sample is less than a predetermined amount.
